(19) United States
(12) Patent Application Publication
(10) Pub. No.: US 2024/0306736 A1
(43) Pub. Date: Sep. 19, 2024

(54) CATAMENIAL UNDERGARMENT DEVICE AND METHOD

(71) Applicant: Frenesha Knight, Pittsburgh, PA (US)

(72) Inventor: Frenesha Knight, Pittsburgh, PA (US)

(21) Appl. No.: 18/123,291

(22) Filed: Mar. 19, 2023

(51) Int. Cl. A41B 9/04; A61F 13/74

(52) U.S. Cl. CPC A41B 9/04; A61F 13/74

(57) ABSTRACT

A catamenial undergarment device and method for facilitating retaining a menstruation pad in a fixed position against a crotch of a wearer includes an underpants garment including a pelvic covering section providing coverage from the waist to a top of the pair of thighs of the wearer. A leg section is coupled to and extends downwardly from a bottom side of the pelvic covering section and provides coverage from the top to a bottom of the pair of thighs of the wearer. A liner is coupled to the underpants garment and facilitates holding the menstruation pad within the underpants garment. A padded layer is coupled to the liner and removably abuts the menstruation pad.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to underpants garments and more particularly pertains to a new underpants garment for facilitating retaining a menstruation pad in a fixed position against a crotch of a wearer.

### (2) Description of Related Art Including Information Disclosed Under 37 CFR 1.97 and 1.98

The prior art relates to underpants garments having a catamenial padding attached to the garment. Known prior art does not include a liner attached to the underpants garment and the catamenial padding being attached to the liner such that the liner facilitates retaining the catamenial padding in place while wearing the underpants garment.

### BRIEF SUMMARY OF THE INVENTION

An embodiment of the disclosure meets the needs presented above by generally comprising an underpants garment including a pelvic covering section configured to provide coverage from the waist to a top of the pair of thighs of the wearer. A leg section is coupled to and extends downwardly from a bottom side of the pelvic covering section and is configured to provide coverage from the top to a bottom of the pair of thighs of the wearer. A liner is coupled to the underpants garment and is configured to facilitate holding the menstruation pad within the underpants garment. A padded layer is coupled the liner and is configured for removably abutting the menstruation pad, the padded layer is fluidly absorbent, wherein the padded layer defines an aid for a menstruation pad.

Another embodiment of the disclosure comprises a method including the steps of abutting a menstruation pad to a crotch of the wearer. A pair of legs of the wearer is inserted through a waist opening of an underpants garment. Each of the legs is inserted through one of a pair of leg openings of the underpants garment. An upper perimeter edge of the underpants garment is positioned around a waist of the wearer. Each of the thighs of the wearer is positioned within one of a pair of sleeves of the underpants garment and a crotch section of a liner is positioned against a crotch of the wearer.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front view of a catamenial undergarment device and method according to an embodiment of the disclosure.

FIG. 2 is a rear view of an embodiment of the disclosure.

FIG. 3 is a top isometric view of an embodiment of the disclosure.

FIG. 4 is an in-use view of an embodiment of the disclosure.

### DETAILED DESCRIPTION OF THE INVENTION

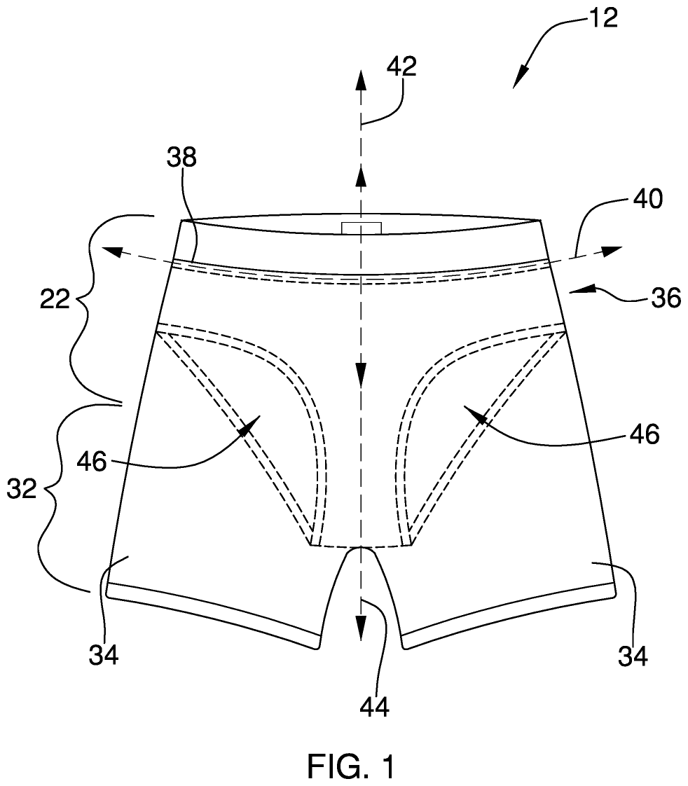

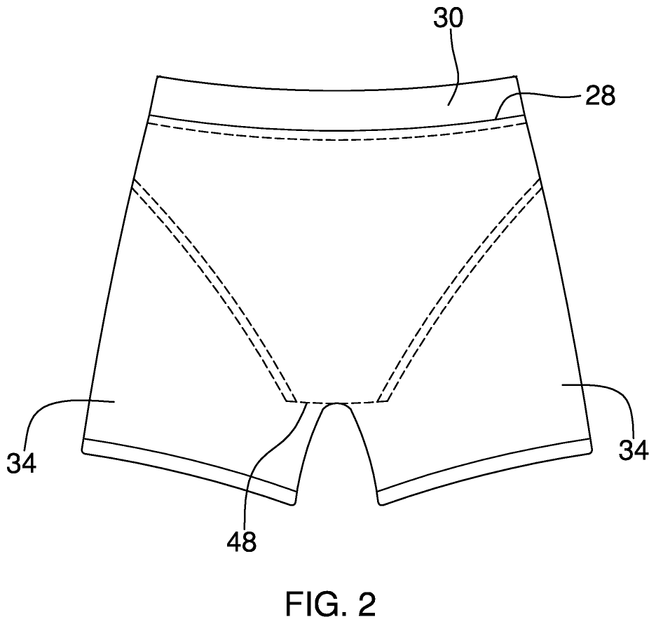

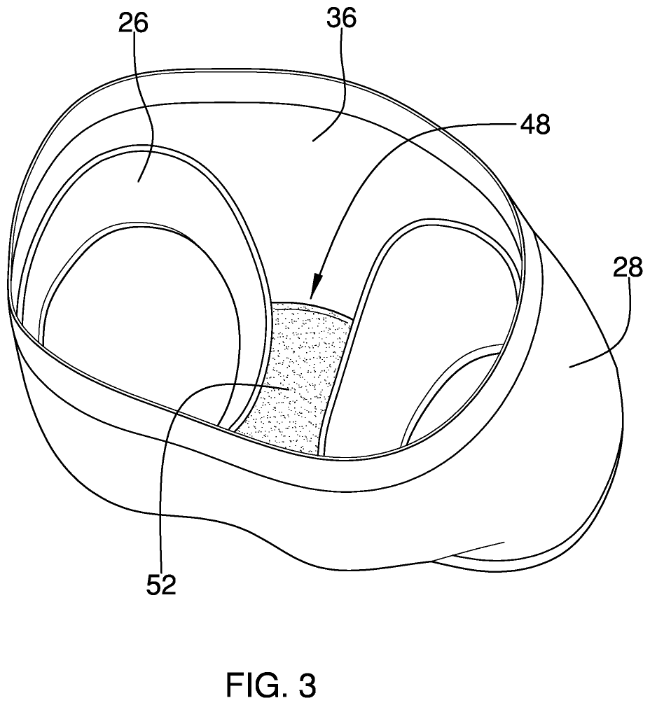

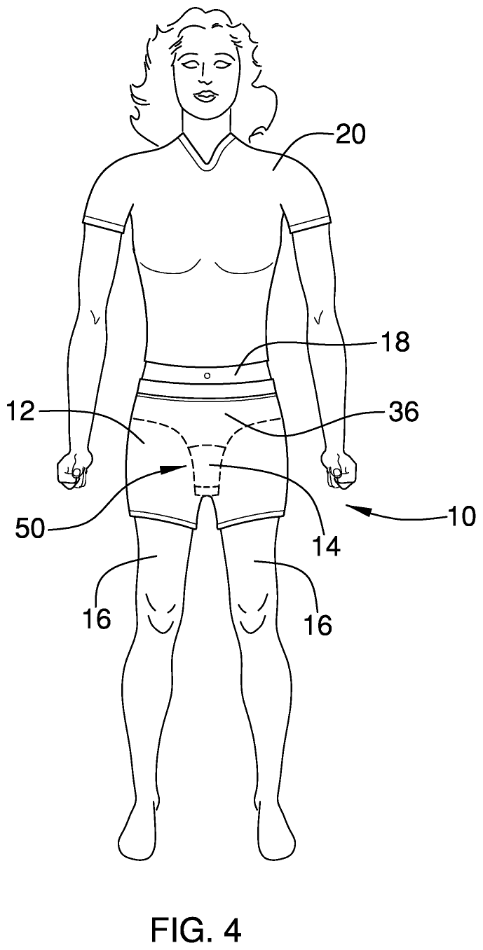

With reference now to the drawings, and in particular to FIGS. 1 through 4 thereof, a new underpants garment embodying the principles and concepts of an embodiment of the disclosure and generally designated by the reference numeral 10 will be described.

As best illustrated in FIGS. 1 through 4, the catamenial undergarment device 10 generally comprises an underpants garment 12 configured to facilitate retaining a menstruation pad 14 in a fixed position between a pair of thighs 16 of a wearer 20 and below a waist 18 of the wearer 20. The underpants garment 12 includes a pelvic covering section 22 configured to provide coverage from the waist 18 to a top of the pair of thighs 16 of the wearer 20. The pelvic covering section 22 includes an exterior surface 24 and an interior surface 26. A top side of the pelvic covering section 22 normally has a waist opening extending therethrough, wherein the waist opening forms an upper perimeter edge 28 between the exterior surface 24 and the interior surface 26 of the pelvic covering section 22 thereon. A waist band 30 may be attached to the upper perimeter edge 28 and may be a resiliently elastic material configured to contour to the waist 18 of the wearer 20. The waist band 30 is configured to facilitate retaining the underpants garment 12 to the wearer 20.

A leg section 32 is coupled to and extends downwardly from a bottom side of the pelvic covering section 22. The leg section 32 is configured to provide coverage from the top to a bottom of the pair of thighs 16 of the wearer 20. The leg section 32 typically comprises a pair of sleeves 34 to define the underpants garment 12 as boxers. Each of the sleeves 34 is coupled to the exterior surface 24 of the pelvic covering section 22 and is elongated therefrom. The bottom side of the pelvic covering section 22 has a pair of leg openings extending therethrough such that each of the leg openings extends through the interior surface 26 and exterior surface 24 of the pelvic covering section 22 into one of the sleeves 34 of the leg section 32. Each of the sleeves 34 is configured to position around one of the pair of thighs 16 of the wearer 20. Each of the sleeves 34 may be the resiliently elastic material configured to contour to one of the pair of thighs 16 of the wearer 20 and to define the underpants garment 12 as boxer briefs.

A liner 36 is coupled to the underpants garment 12 and is configured to facilitate holding the menstruation pad 14 within the underpants garment 12. The liner 36 normally includes a boundary edge 38 attached to the interior surface 26 of the pelvic covering section 22 proximate to the upper perimeter edge 28. A horizontal plane 40 of the boundary edge 38 of the liner 36 is perpendicular to a longitudinal axis 42 of the waist opening. A vertical axis 44 of the liner 36 is co-axial to the longitudinal axis 42 of the waist opening. The liner 36 normally has a pair of holes 46 extending therethrough. Each of the thighs 16 of the wearer 20 is configured to position simultaneously through one of the pair of holes 46 and within one of the pair of sleeves 34. An area between each of the holes 46 defines a crotch section 48, wherein the crotch section 48 is configured for positioning adjacent to a crotch 50 of the wearer 20.

A padded layer 52 is coupled to the liner 36 and is configured for removably abutting the menstruation pad 14. The padded layer 52 is normally mounted to the crotch section 48 of the liner 36. The padded layer 52 may be fluidly absorbent, wherein the padded layer 52 may define an aid for a menstruation pad 14. The padded layer 52 may act as a catamenial padding to absorb menstruation discharge if the menstruation pad 14 is not abutting the crotch 50 of the wearer 20.

In use, the menstruation pad 14 is abutted to the crotch 50 of the wearer 20. The pair of thighs 16 of the wearer 20 is inserted through the waist opening of the underpants garment 12. Each of the thighs 16 is inserted through one of the pair of holes 46 the liner 36 prior to each of the thighs 16 inserting through one of the pair of leg openings of the underpants garment 12. The upper perimeter edge 28 of the underpants garment 12 is positioned around the waist 18 of the wearer 20 and each of the thighs 16 of the wearer 20 is positioned within one of the pair of sleeves 34 of the underpants garment 12. The crotch section 48 of the liner 36 is positioned against the crotch 50 of the wearer 20 such that the padded layer 52 is abutting the menstruation pad 14 positioned abutting the crotch 50.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A feminine undergarment device being configured to facilitate retaining a menstruation pad in a fixed position between a pair of thighs of a wearer and below a waist of the wearer, said feminine undergarment comprising:
an underpants garment including:
a pelvic covering section configured to provide coverage from the waist to a top of the pair of thighs of the wearer;
a leg section being coupled to and extending downwardly from a bottom side of said pelvic covering section, said leg section being configured to provide coverage from the top to a bottom of the pair of thighs of the wearer; and
a liner being coupled to said underpants garment, said liner being configured to facilitate holding the menstruation pad within said underpants garment; and
a padded layer being coupled to said liner and being configured for removably abutting the menstruation pad, said padded layer being fluidly absorbent, wherein said padded layer defines an aid for a menstruation pad.

2. The feminine undergarment of claim 1, wherein said pelvic covering section includes an exterior surface and an interior surface, a top side of said pelvic covering section having a waist opening extending therethrough, wherein said waist opening forms an upper perimeter edge between said exterior surface and said interior surface of said pelvic covering section thereon.

3. The feminine undergarment of claim 2, wherein said leg section comprises a pair of sleeves, each of said sleeves being coupled to said exterior surface of said pelvic covering section and being elongated therefrom, said bottom side of said pelvic covering section having a pair of leg openings extending therethrough, each of said leg openings extending through said interior and exterior surface of said pelvic covering section into one of said sleeves of said leg section, each of said sleeves being configured to position around one of the pair of thighs of the wearer.

4. The feminine undergarment of claim 2, further including a waistband being attached to said upper perimeter edge, said waistband being a resiliently elastic material configured to contour to the waist of the wearer.

5. The feminine undergarment of claim 2, wherein said liner includes boundary edge, said boundary edge being attached to said interior surface of said pelvic covering section.

6. The feminine undergarment of claim 1, wherein a center axis of each of said holes is co-axial to a middle axis of one of said pair of sleeves, wherein each of the thighs of the wearer is configured to position simultaneously through one of said pair of holes and within one of said pair of sleeves, an area between each of said holes defining a crotch section, wherein said crotch section is configured for positioning adjacent to a crotch of the wearer.

7. The feminine undergarment of claim 6, wherein said padded layer being mounted to said crotch section of said liner.

8. A feminine undergarment device being configured to facilitate retaining a menstruation pad in a fixed position between a pair of thighs of a wearer and below a waist of the wearer, said feminine undergarment comprising:
an underpants garment including:
a pelvic covering section configured to provide coverage from the waist to a top of the pair of thighs of the wearer, said pelvic covering section including an exterior surface and an interior surface, a top side of said pelvic covering section having a waist opening extending therethrough, wherein said waist opening forms an upper perimeter edge between said exterior surface and said interior surface of said pelvic covering section thereon;
a leg section being coupled to and extending downwardly from a bottom side of said pelvic covering section, said leg section being configured to provide coverage from the top to a bottom of the pair of thighs of the wearer, said leg section comprising a pair of sleeves, each of said sleeves being coupled to said exterior surface of said pelvic covering section and being elongated therefrom, said bottom side of said pelvic covering section having a pair of leg openings extending therethrough, each of said leg openings extending through said interior and exterior surface of said pelvic covering section into one of said sleeves of said leg section, each of said sleeves being configured to position around one of the pair of thighs of the wearer; and
a waistband being attached to said upper perimeter edge, said waistband being a resiliently elastic material configured to contour to the waist of the wearer;
a liner being coupled to said underpants garment, said liner being configured to facilitate holding the menstruation pad within said underpants garment, said liner including boundary edge, said boundary edge being attached to said interior surface of said pelvic covering section, said liner having a pair of holes extending therethrough, a center axis of each of said holes being co-axial to a middle axis of one of said pair of sleeves, wherein each of the thighs of the wearer is configured to position simultaneously through one of said pair of holes and within one of said pair of sleeves, an area between each of said holes defining a crotch section, wherein said crotch section is configured for positioning adjacent to a crotch of the wearer; and
a padded layer being coupled to said liner and being configured for removably abutting the menstruation pad, said padded layer being mounted to said crotch section of said liner, said padded layer being fluidly absorbent, wherein said padded layer defines an aid for a menstruation pad.

9. A method for securing feminine products in place relative to a wearer comprising the steps of:
abutting a menstruation pad to a crotch of the wearer;
inserting a pair of thighs of the wearer through a waist opening of an underpants garment;
inserting each of said thighs through one of a pair of leg openings of said underpants garment;
positioning an upper perimeter edge of said underpants garment around a waist of the wearer;
positioning each of the thighs of the wearer within one of a pair of sleeves of said underpants garment; and
positioning a crotch section of a liner against a crotch of the wearer.

10. The method of claim 9, wherein the step of inserting each of said thighs through one of a pair of leg openings of said underpants garment further includes the step of inserting each of said thighs through one of a pair of holes of said liner.
